(19) United States
(12) Patent Application Publication
(10) Pub. No.: US 2019/0345121 A1
(43) Pub. Date: Nov. 14, 2019

(54) PYRENE-BASED COMPOUNDS FOR INTRACELLULAR ORGANELLE IMAGING APPLICATIONS

(71) Applicants: Yi Pang, Copley, OH (US); Chathura Shalitha Abeywickrama, Akron, OH (US)

(72) Inventors: Yi Pang, Copley, OH (US); Chathura Shalitha Abeywickrama, Akron, OH (US)

(21) Appl. No.: 16/409,984

(22) Filed: May 13, 2019

Related U.S. Application Data
(60) Provisional application No. 62/670,382, filed on May 11, 2018.

(51) Int. Cl. C07D 277/64; C07D 209/04; C07D 213/06

(52) U.S. Cl. CPC C07D 277/64; G01N 33/52; C07D 213/06; C07D 209/04

(57) ABSTRACT

A pyrene-based compound for imaging intracellular organelles, where the pyrene-based compound is defined by a formula selected from the group consisting of:

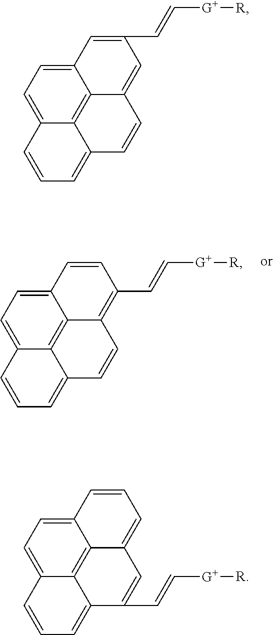

The G⁺ of the pyrene-based compound is selected from the group consisting of a pyridinium or a benzothiazolium group. The R group of the pyrene-based compound is selected from the group consisting of a methyl group, an ethyl group, a propyl group or a benzyl group.

### CROSS-REFERENCE TO RELATED APPLICATIONS

The present application claims priority to U.S. Provisional Patent Application No. 62/670,382 entitled “Bright Fluorescent Pyrene Derivatives with a Large Stokes Shift for Cell Nucleus Staining” filed May 11, 2018 and U.S. Provisional Patent Application No. 62/806,987 entitled “Red-Emitting Pyrene-Benzothiazolium: Unexpected Selectivity to Lysosomes for Real-Time Cell Imaging” filed Feb. 18, 2019, both of which are incorporated herein by reference.

### FIELD OF THE INVENTION

The present invention relates to a biological imaging agent. The present invention further relates to pyrene-based imaging agents that can recognize intracellular organelles. The present invention further relates to pyrene-based fluorescent dyes that are found to exhibit high selectivity for specific intracellular organelles while having emission peaks above 600 nm, high fluorescence quantum yields, and a large Stokes shift, properties which allows the staining to be achieved at low probe concentrations.

### BACKGROUND OF THE INVENTION

For biological imaging studies, one of the most important tasks is to stain nuclei of live cells in order to distinguish cellular organelles. For example, visualization of the cell nucleus is widely used in the study of cell growth and development, fluorescent probe co-localization, DNA quantification, and cancer biology. Among the dyes for live-cell nuclear imaging, the most commonly used are DAPI (λₑₓ=358 nm, λₑₘ=461 nm) and Hoechst (λₑₓ=352 nm, λₑₘ=461 nm). However, these dyes require UV excitation, which can cause damage to DNA and cells. Although DRAQ5 (λ_{abs}=676 nm, λₑₓ=616 nm, λₑₘ=681) has been known to emit a deep red emission, it has a small Stokes shift and low fluorescence quantum yield (ϕ_{fl} of about 0.003 in the presence of DNA in Tris buffer).

Since the discovery of lysosomes in 1955, the organelles are known to contain a variety of digestive enzymes called “hydrolases”, which exhibit optimal activity in a narrow acidic pH environment within the lysosomal lumen (pH 4.5-6). Abnormal lysosome activity or lysosomal malfunctions have been attributed to many disease conditions including neurodegenerative disorders, lysosome storage disease, cancer and inflammations. Imaging lysosomes in live or fixed cell samples has recently received attention due to their involvement in cell processing activities and cancer symptoms. Many probe designs incorporate basic amino groups for lysosome selectivity which includes LysoTracker® Green DND-26 (λₑₓ=504 nm, λₑₘ=510) and LysoTracker® Red DND-99(λₑₓ=577 nm, λₑₘ=590 nm). Since the existing fluorescent probes require an acidic media to operate, their accumulation in lysosomes will cause the pH to increase (an “alkalinizing effect”) and affects the normal cell activity. Although new lysosome probes have been developed with spirolactams and peptide based molecules, protonation of an amine group remains an essential step.

Most existing DNA probes, such as DRAQ5, suffer from having a small Stokes shift, which prevents the optimum use of fluorescence signals, and thereby lowers the sensitivity of the dye. There have also been attention shown in the field of developing new probes that exhibit emission near the infrared (NIR) region. However, no intracellular organelle probes exist that exhibit these qualities, therefore, there is a need in the art to develop fluorescent dyes that exhibit long-wavelength emission with a large Stokes shift, high selectivity and sensitivity to nucleic acids, and low cytotoxicity for live cell imaging.

### SUMMARY OF THE INVENTION

In a first embodiment, the present invention provides a pryrene based compound for imaging intracellular organelles, where the pyrene-based compound is defined by a formula selected from the group consisting of:

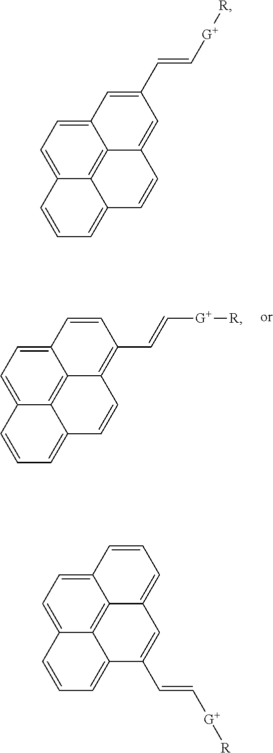

wherein G⁺ is selected from the group consisting of a pyridinium or a benzothiazolium group and wherein R is selected from the group consisting of a methyl group, an ethyl group, a propyl group or a benzyl group.

In a second embodiment, the present invention provides a pyrene-based compound as in any embodiment above, wherein the pyridinium group is selected from the group consisting of 4-alkylpyridinium or 2-alkylpyridinium.

In a third embodiment, the present invention provides a pyrene-based compound as in any embodiment above, wherein the pyrene-based compound is used to image a nucleus of a cell.

In a fourth embodiment, the present invention provides a pyrene-based compound as in any embodiment above, wherein the benzothiazolium group is selected from the group consisting of 3-alkylbenzothiazolium, 3-alkylbenzooxazolium, 1-methyl-3alkylbenzoimidazolium, or 3,3-dimethyl-1-alkylindolium.

In a fifth embodiment, the present invention provides a pyrene-based compound as in any embodiment above, wherein the pyrene-based compound is used to image the lysosomes of a cell.

In a sixth embodiment, the present invention provides a pyrene-based compound as in any embodiment above, wherein the pyrene-based compound exhibits a bright red emission of between about 600 nm and about 650 nm when excited via a one-photon absorption process at about 475 nm.

In a seventh embodiment, the present invention provides a pyrene-based compound as in any embodiment above, wherein the pyrene-based compound exhibits a bright red emission of between about 600 nm and about 650 nm when excited via a two-photon absorption process upon excitation by about a 950 nm laser.

In an eighth embodiment, the present invention provides a pyrene-based compound as in any embodiment above, wherein the pyrene-based compound has a Stokes' shift of greater than about 130 nm.

In a ninth embodiment, the present invention provides a pyrene-based compound as in any embodiment above, wherein the pyrene-based compound has a LC₅₀ of at least 8 μM.

In a tenth embodiment, the present invention provides a method of producing a pyrene-based compound, the method comprising the steps of: adding a benzothiazolium salt or pyridinium salt to a pyrene-carbaldehyde to form a solution; adding a reaction promotor to the solution; and heating the solution so as to produce a pyrene-based compound defined by a formula selected from the group consisting of:

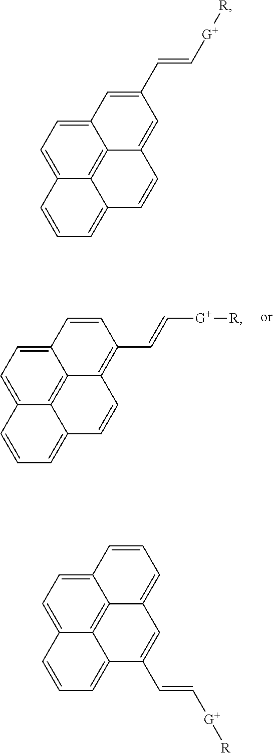

wherein G⁺ is selected from the group consisting of a pyridinium or a benzothiazolium group and wherein R is selected from the group consisting of a methyl group, an ethyl group, a propyl group or a benzyl group.

In a eleventh embodiment, the present invention provides a method of producing a pyrene-based compound as in any embodiment above, wherein a benzothiazolium salt is utilized and the benzothiazolium salt is selected from the group consisting of 3-alkyl-2-methylbenzothiazol-3-ium halides.

In a twelfth embodiment, the present invention provides a method of producing a pyrene-based compound as in any embodiment above, wherein the alkyl group is selected from the group consisting of methyl, ethyl, propyl, and benzyl; and wherein the halide group is selected from the group consisting of chloride, bromide, and iodide.

In a thirteenth embodiment, the present invention provides a method of producing a pyrene-based compound as in any embodiment above, wherein a pyridinium salt is utilized and the pyridinium salt is selected from the group consisting of 1-alkyl-4-methylpyridinium halides.

In a fourteenth embodiment, the present invention provides a method of producing a pyrene-based compound as in any embodiment above, wherein the alkyl group is selected from the group consisting of methyl, ethyl, propyl and benzyl; and wherein the halide group is selected from the group consisting of chloride, bromide, and iodide.

In a fifteenth embodiment, the present invention provides a method of producing a pyrene-based compound as in any embodiment above, wherein the pyrene-carbaldehyde is selected from the group consisting of pyrene-1-carboxaldehyde, pyrene-2-carboxaldehyde, and pyrene-4-carboxaldehyde.

In a sixteenth embodiment, the present invention provides a method of producing a pyrene-based compound as in any embodiment above, wherein the reaction promoter is selected from the group consisting of pyridine, piperidine, and triethylamine.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates fluorescent confocal microscopy images of COS-7, A549, Huh 7.5 and HEK 293 cells treated with probes 2a-2d made from pyrene-based compounds having a benzothiazolium group of the present invention (500 nM) for 30 minutes;

FIG. 2 illustrates a series of fluorescent confocal microscopy images, wherein column 1 shows COS-7 cells stained with LysoTracker® Green DND-26 (488 nM), column 2 shows COS-7 cells stained with probes 2a-2d made from pyrene-based compounds having a benzothiazolium group of the present invention (561 nM), and column 3 shows overlapped images of columns 1 and 2;

FIG. 3 illustrates Mander's correlation coefficient calculation for probes 2a-2d made from pyrene-based compounds having a benzothiazolium group of the present invention for colocalization studies conducted with LysoTracker® Green DND-26;

FIG. 4A shows fluorescence microscopy images obtained from a probe made from pyrene-based compounds having a benzothiazolium group of the present invention in A549 cells over a period of 6 hours;

FIG. 4B shows fluorescence microscopy images obtained for LysoTracker® Red DND-99 in A549 cells over a period of 3 hours;

FIG. 5 shows a fluorescence confocal image obtained for COS-7 cells incubated with a probe made from pyrene-based compounds having a benzothiazolium group of the present invention taken after a 30 minute period (a) and after a 24 hour period (b);

FIG. 6 shows fluorescent confocal microscopy images of a probe made from pyrene-based compounds having a pyridinium group of the present invention (500 nM) treated with COS-7 cells for 30 minutes, wherein image (b) shows a zoomed-in view of the selected area in image (a);

FIG. 7 illustrates a series of fluorescent confocal microscopy images, wherein column 1 shows fluorescent confocal microscopy images of COS-7 cells stained with commercial pmTurquoise-H2A for 14 hours; column 2 shows fluorescent confocal microscopy images of COS-7 cells stained with a probe made from pyrene-based compounds having a pyridinium group of the present invention for 30 minutes at 500 nM; column 3 shows overlapped images of the images of column 1 with the images of column 2; and the images of row 2 show a digitally magnified view of the selected region in the corresponding image of row 1;

FIG. 8A shows spectra recorded for pyrene-based compounds having a pyridinium group (1×10-5 M) in CH₂Cl₂ at room temperature (excitation between 850 nm-1150 nm); and

FIG. 8B shows a normalized two-photon excitation spectrum by plotting fluorescence intensity at 610 nm (normalized) while exciting between 850-1050 nm.

### DETAILED DESCRIPTION OF ILLUSTRATIVE EMBODIMENTS

In one or more embodiments, the present invention relates to pyrene-based imaging agents that can recognize intracellular organelles. In other embodiments, the present invention relates to pyrene-based imaging agents containing a pyridinium group or a benzothiazolium group. In yet other embodiments, the pyrene-based imaging agents containing a pyridinium group are used, in particular, to image the nucleus of a cell and the pyrene-based imaging agents containing a benzothiazolium group are used, in particular, to image the lysosomes of a cell.

In one or more embodiments, the pyrene-based imaging agents of the present invention are defined by a formula selected from the group consisting of:

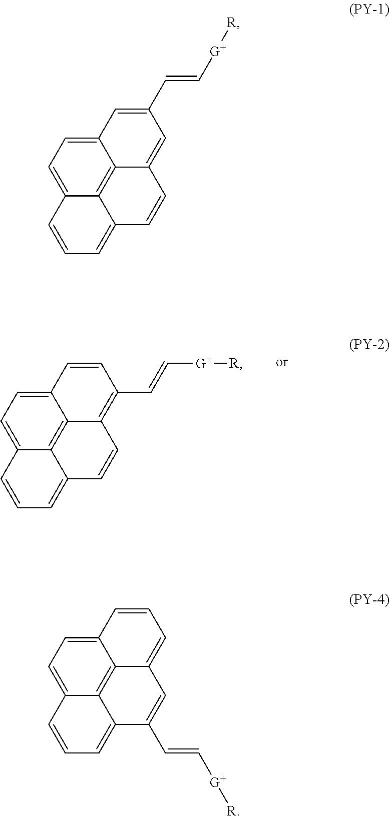

In one or more embodiments of the present invention, the G⁺ group as shown on PY-1, PY-2, and PY-4 is selected from the group consisting of a pyridinium or a benzothiazolium group.

In one or more embodiments of the present invention, G⁺ is a pyridinium group selected from the group consisting of 4-alkylpyridinium or 2-alkylpyridinium. Wherein 4-alkylpyridinium is represented by the formula

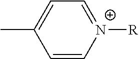

and wherein 2-alkylpyridinium is represented by the formula

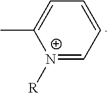

In one or more embodiments of the present invention, G⁺ is a benzothiazolium group selected from the group consisting of 3-alkylbenzothiazolium, 3-alkylbenzooxazolium, 1-methyl-3-alkylbenzoimidazolium, or 3,3-dimethyl-1-alkylindolium. Wherein 3-alkylbenzothiazolium is represented by the formula

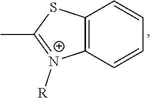

wherein 3-alkylbenzooxazolium is represented by the formula

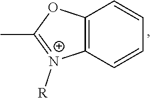

wherein 1-methyl-3alkylbenzoimidazolium is represented by the formula

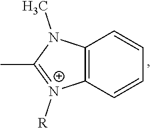

and wherein 3,3-dimethyl-1-alkylindolium is represented by the formula

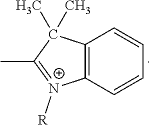

In one or more embodiments of the present invention, the R group as shown on PY-1, PY-2, and PY-4, and as more specifically shown on the various specific G⁺ groups, is selected from the group consisting of a methyl group, an ethyl group, a propyl group or a benzyl group.

In one or more embodiments of the present invention, if G⁺ is a pyridinium group, then the pyrene-based imaging agents are utilized so as to image the nucleus of a cell. In one or more embodiments of the present invention, if G⁺ is a benzothiazolium group, then the pyrene-based imaging agents are utilized so as to image the lysosomes of a cell.

In one or more embodiments, the pyrene-based imaging agents of the present invention can be prepared through the following process. A benzothiazolium salt or pyridinium salt is added to a solution of a pyrene-carbaldehyde in a solvent. The resultant solution is then stirred at room temperature for 5 minutes. Then, 0.2 mL of a reaction promoter is added. This solution is heated to 65° C. and stirred for 24 hours. After completion of the reaction, the mixture is cooled down to room temperature and concentrated under a vacuum. To the resulting solid, 10 mL of a solvent is added and the solution is left to settle for 30 minutes. Then, the solid is collected by vacuum filtration and washed with 10 mL of an ethanol based solvent three times

In one or more embodiments, the pyrene-carbaldehyde used in the method of making the pyrene-based imaging agents of the present invention are selected from the group consisting of pyrene-1-carboxaldehyde, pyrene-2-carboxaldehyde, and pyrene-4-carboxaldehyde.

In one or more embodiments, the reaction promoter used in the method of making the pyrene-based imaging agents of the present invention are selected from the group consisting of pyridine, piperidine, and triethylamine.

In one or more embodiments, the solvent used prior to vacuum filtration is selected from the group consisting of ethyl acetate, diethyl ether, tetrahydrofuran, and methylene chloride.

In one or more embodiment, the solvent used after the solid has been collected by vacuum filtration is selected from the group consisting of ethyl acetate and diethyl ether.

In one or more embodiments of the present invention, the pyrene-based compounds of the present invention have bright red emission of greater than about 600 nm. In one or more embodiments of the present invention, the pyrene-based compounds of the present invention have bright red emission of between about 600 nm and about 650 nm, in other embodiments between about 615 nm and about 645 nm, and in yet other embodiments between about 630 nm and about 640 nm.

In one or more embodiments of the present invention, the pyrene-based compounds of the present invention have a large Stokes' shift Δλ of greater than about 120 nm, in other embodiments of greater than about 125 nm, and in yet other embodiments greater than about 130 nm.

The pyrene-based compounds of the present invention exhibit low cytotoxicity. In one embodiment of the present invention, the pyrene-based compounds having a benzothiazolium group of the present invention also exhibit low cytotoxicity. Specifically, in some embodiments of the present invention the pyrene-based compounds having a benzothiazolium group of the present invention exhibit an LC₅₀ of greater than 15 μM, in other embodiments greater than 20 μM, and in yet other embodiments greater than 25 μM. In another embodiment of the present invention, the pyrene-based compounds having a pyridinium group has LC₅₀ of greater than 8 μM.

The pyrene-based compounds of the present invention also exhibit a large fluorescence turn on which allows the pyrene-based compounds of the present invention to enable wash-free staining. Having a wash-free staining characteristic is important because it allows for quick imaging immediately after the incubation of the dye, without the need for removing the excess dyes prior to imaging.

The pyrene-based compounds of the present invention also exhibit stable emission throughout a wide pH range (1.0-12.0), as the probe structure is insensitive to acidity. By eliminating the “alkalinizing effect”, probes made from the pyrene-based compounds of the present invention have significant advantages over existing commercial probes, and can be used for long term tracking of intracellular organelle activity. Since probes made from the pyrene-based compounds of the present invention do not exhibit an “alkalinizing effect” and exert minimal perturbation on the digestive activities of enzymes, they are valuable tools for monitoring intracellular organelle activity in biological research.

### EXPERIMENTAL

The following examples are offered to more fully illustrate the invention, but are not to be construed as limiting the scope thereof. Further, while some of examples may include conclusions about the way the invention may function, the inventors do not intend to be bound by those conclusions, but put them forth only as possible explanations. Moreover, unless noted by use of past tense, presentation of an example does not imply that an experiment or procedure was, or was not, conducted, or that results were, or were not actually obtained. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

All chemicals for synthesis were purchased from Sigma-Aldrich, Fisher Scientific, or Acros Organics. All molecular biology grade reagents for cell culture and fluorescent confocal microscopy were purchased from Sigma-Aldrich, Fisher Scientific, Abcam, or Addgene. UV-vis studies were carried out on a Hewlett Packard-8453 diode array spectrophotometer at 25° C. Fluorescence studies were carried out on a HORIBA Fluoromax-4 spectrofluorometer. Fluorescence confocal images were obtained from a Zeiss LSM 710 confocal microscope. Cell viability assays were carried out by use a Spectramax® M5e multimode microplate reader. Cercopithecus aethiops kidney cells (COS-7), Adeno-carcinomic human alveolar basal epithelial cells (A549), human hepatocyte-derived carcinoma cells (Huh 7.5) or Human embryonic kidney cells (HEK 293) were used for cell imaging studies.

### Experiment 1

1 mmol of a benzothiazolium salt was added to a solution of 1.1 mmol pyrene-1-carboxaldehyde in 15 mL of methanol. The solution was stirred at room temperature for 5 minutes. Then, 0.2 mL of pyridine was added so as to promote the reaction. This solution was heated to 65° C. and stirred for 24 hours. After completion of the reaction, the mixture was cooled down to room temperature and concentrated under vacuum. To the resulting brown colored solid, 10 mL of ethyl acetate was added and the solution was left to settle for 30 minutes. Then, the brown colored solid was collected by vacuum filtration and washed with 10 mL of ethyl acetate three times. This experiment was done multiple times with various benzothiazolium salts and the final products were collected as a fine dark brown powder with between a 68-76% yield.

### Fluorescence Quantum Yields

The fluorescence quantum yields for the final products were calculated using Rhodamine 6G as the standard where the quantum yield for Rhodamine 6G is 0.95 in ethanol at 490 nm. It was found that the fluorescent quantum yield of the pyrene-based compounds having a benzothiazolium group of the present invention was lower in an aqueous environment (ϕ_{fl}≈0.05) than that in a non-aqueous media, making it possible for cell staining under “wash-free” conditions.

### Cell Culture

For the cell culture aspect of the experiment, COS-7 and HEK 293 cells were maintained in Dulbecco's modified Eagle's medium (DMEM) containing 1% FBS and 1% Penstrep at 37° C. in a 5% CO₂ humidified incubator. A549 and Huh cells were maintained in Roswell Park Memorial Institute medium (RPMI) containing 1% FBS and 1% Penstrep at 37° C. in a 5% CO₂ humidified incubator. The cells were grown to 70 to 80% confluence before passing or transfection. Live cell imaging plates were prepared by plating cells in MatTek chambered cell culture plates.

Cell Treatment with Fluorescent Probes

For the cell treatment with fluorescent probes aspect of the experiment, probes were made using the pyrene-based compounds having a benzothiazolium group of the present invention, LysoTracker® Red DND-99, LysoTracker® Green DND-26, and MitoTracker® Green FM solutions made in DMSO, respectively. For live cell imaging, cells were treated with 100-500 nM probes using solutions of the pyrene-based compounds having a benzothiazolium group of the present invention in PBS for 30 minutes at 37° C. During co-localization studies, cells were treated with 70 nM LysoTracker® Red or LysoTracker® Green. Final concentration of the MitoTracker® Green was maintained at 200 nM concentration for co-localization studies. Final DMSO percentages in the cell media were less than 0.25% (V/V). For the initial cell studies, cells treated with the probes made from the pyrene-based compounds having a benzothiazolium group of the present invention were used for fluorescent confocal imaging without further washing. During co-localization studies with the LysoTracker® or MitoTracker® probes, the cells were washed with PBS 3 times.

### Fluorescence Confocal Microscopy

For the fluorescence confocal microscopy aspect of the experiment, cells were imaged using a Zeiss LSM 710 fluorescence confocal microscope with an oil 63×1.4 numerical aperture objective. LysoTracker® Green DND-26 and MitoTracker® Green-FM were excited with a 488 nm laser and emissions were collected from the 500 nm to 580 nm range. Probes using solutions of the pyrene-based compounds having a benzothiazolium group of the present invention and LysoTracker® Red DND-99 were excited with a 561 nm laser and emissions were collected from the 585 nm to 700 nm range. Co-localization experiment results were further analyzed by using Zeiss LSM software and Mander's overlap coefficient was calculated by ImageJ (NIH) software. A Mander's overlap coefficient value of greater than 0.6 confirms an acceptable significant co-localization of the two fluorophores.

### Long-Term Imaging Ability

For the long-term imaging ability assessment aspect of the experiment, a Zeiss LSM 710 fluorescence confocal microscope with identical settings were used for photostability comparisons of the probes (561 nm (15 mW) laser line; Laser power percentage 1.0; Digital zoom=1; Pinhole=1A U; Master Gain=500; Digital offset=0). Different imaging plates with A549 cells were incubated for 30 minutes at 70 nm with probes made using solutions of the pyrene-based compounds having a benzothiazolium group of the present invention as well as with probes made from LysoTracker® Red DND-99. Fluorescence confocal microscopy images were obtained at 30-minute intervals (identical fluorescence microscopy settings were used during the experiments). ImageJ (NIH) software was used to analyze the obtained images and average relative fluorescence intensity was plotted as a function of time.

### Photostability Assessment

For the photostability assessment aspect of the experiment, a Zeiss LSM 710 fluorescence confocal microscope with identical settings was used to compare the photostability of the probes (561 nm (15 mW) laser line; Laser power percentage 3.0; Digital zoom=1; Pinhole=1AU; Master Gain=700; Digital offset=0). Probes made using solutions of the pyrene-based compounds having a benzothiazolium group of the present invention as well as with probes made from LysoTracker® Red were used for the time stability experiment. A549 cells were incubated with the probes for 30 minutes and continuously irradiated with a 561 nm laser. Confocal images of the stained cells were obtained within 10-second intervals over a period of 8 minutes. ImageJ software was used for calculating average fluorescence intensity of the images and a plot was obtained for % intensity of the fluorescence recovered as a function of time.

### Cell Viability

For the cell viability testing aspect of the invention, COS-7 cells were seeded in Corning™ 96 clear well bottom tissue culture treated opaque microplates. Approximately 1000 cells were plated in each well bottom (cell density=1×10⁵ cells/ml). 24-hours after seeding, the cells were treated with increasing concentrations of probe dyes made using solutions of the pyrene-based compounds having a benzothiazolium group of the present invention for 1 hour at 37° C. Cell toxicity assays were performed using a CellTiter-Glow Luminescent® cell viability assay kit (Promega) according to manufacture protocols. Luminescence readings were recorded using Spectramax® 5e microplate readers by using the default software installed. LC50 values for probes were calculated by using dose-response formulas in Origin8 software.

### Lipid Vesicle (MLV) Preparation

For the lipid vesicle (MLV) preparation aspect of the invention, large multilamellar vesicles (MLV's) were prepared (0.2-1 μm diameter range) by using Phosphatidycholine (PC) and Phosphatidylethanolamine (PE) in a chloroform solution. MLV composition was PC:PE (80:20). Appropriate amounts from PC and PE were mixed in a vial maintaining a total lipid concentration of 1 mM. Lipids were hydrated with re-suspension buffer (25 mM HEPES, pH 7.4, containing 150 mM KCl) and incubated at 37° C. for 15 minutes to facilitate hydration of lipids to form while vigorously vortexing MLV's. These lipid solutions were then incubated with 10 μM probes made using solutions of the pyrene-based compounds having a benzothiazolium group of the present invention for 15 minutes. A control experiment was carried out by incubating 10 μM probes made using solutions of the pyrene-based compounds having a benzothiazolium group of the present invention in HEPES buffer (pH 7.4) for 15 minutes. MLV solutions and controls were then transferred to a 96 well opaque bottom assay plate and fluorescence emissions were recorded using a Spectramax® 5e microplate reader by using a 530 nm excitation.

### Results

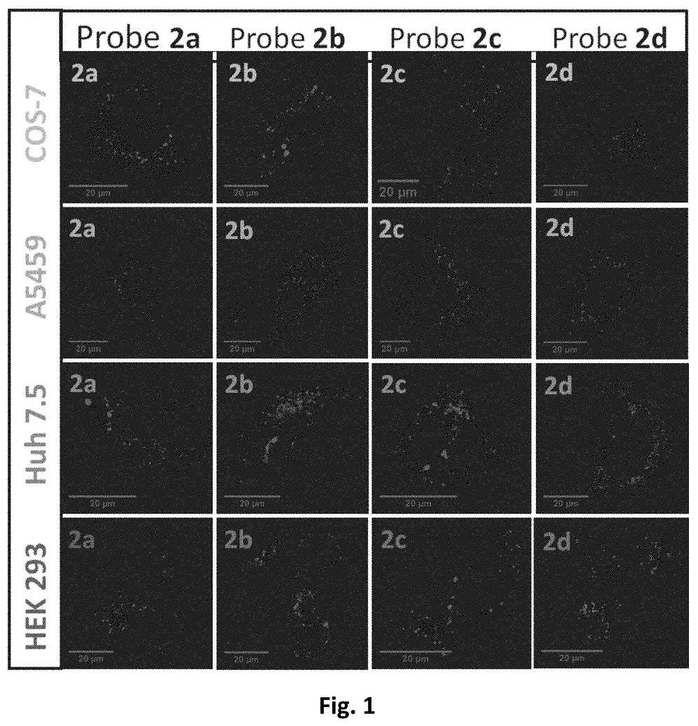

The pyrene-based compounds having a benzothiazolium group of the present invention showed bright red emission with λₑₘ˜630-640 nm (ϕ_{fl}≈0.22-0.48 in DMSO), along with a large Stokes shift Δλ≈140 nm, 4170 cm⁻¹). Bright red emission was observed when using pyrene-based compounds having a benzothiazolium group of the present invention (500 nM), labeled as probes 2a-2d, to stain different cell lines (COS-7, HEK293, A549 and Huh 7.5) as shown in FIG. 1, where the R group on the ammonium nitrogen is selected from methyl (2a), ethyl (2b), propyl (2c), and benzyl substituent (2d). Probes 2a-2d were excited with a 561 nm laser and emission collected in 580 to 720 nm. Clear cell imaging was observable after 30 minutes of staining without any post staining washing.

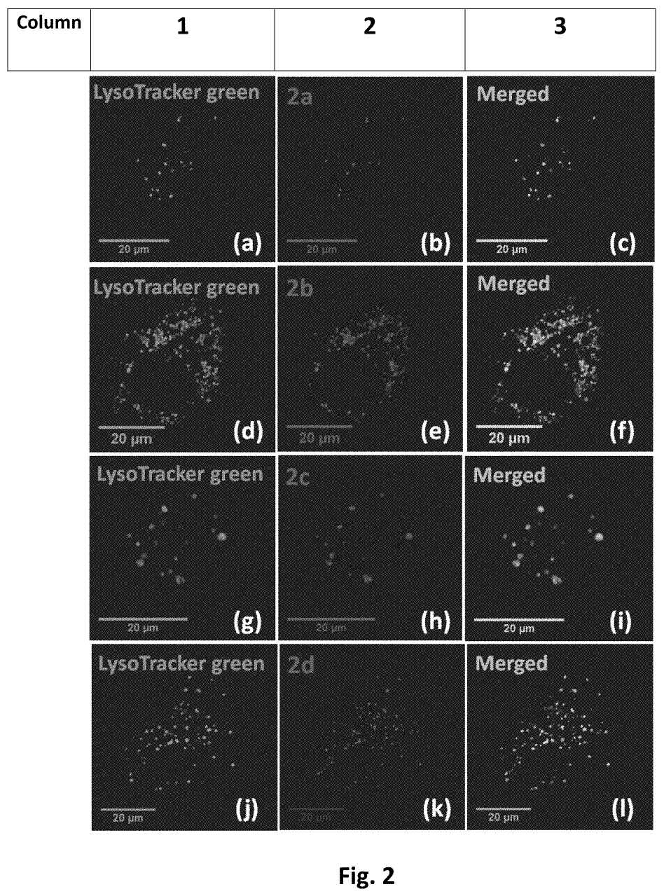

The non-uniform pattern observed from the cell imaging suggested that the dyes might be selectively bound to certain intracellular organelles. The possibility of the dyes binding to mitochondria was ruled out by co-staining the pyrene-based compounds having a benzothiazolium group of the present invention with commercial MitoTracker® Green FM on COS-7 cells. The intracellular location of the pyrene-based compounds having a benzothiazolium group of the present invention was eventually confirmed by co-staining with LysoTracker® Green DND-26, which showed excellent co-localization with probes 2a-2d of the pyrene-based compounds having a benzothiazolium group of the present invention as shown in FIG. 2. Column 1 shows fluorescent confocal microscopy images of COS-7 cells stained with commercial LysoTracker® Green DND-26 (70 nm); column 2 shows fluorescent confocal microscopy images of COS-7 cells stained with probes 2a-2d of the pyrene-based compounds having a benzothiazolium group of the present invention; and column 3 shows overlapped images of the images of column 1 with the images of column 2.

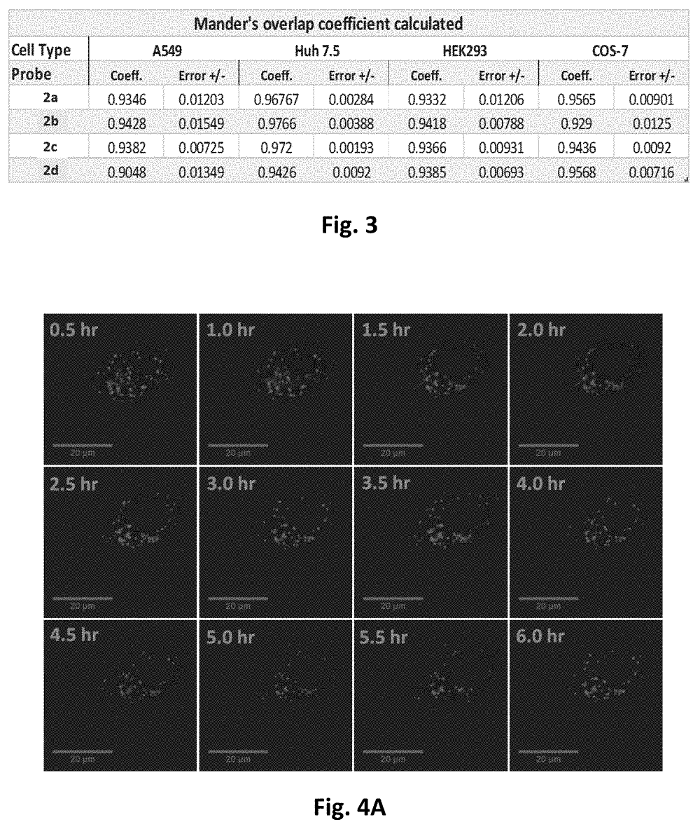

The calculated Mander's correlation coefficients for the pyrene-based compounds having a benzothiazolium group of the present invention were 40.9 in all cell lines, indicating the exceptional lysosome specificity as shown in FIG. 3.

Consistent results were also observed in the co-localization with LysoTracker® Green DND-26 in HEK 293 cells and in cancer cell lines (A549 and Huh 7.5), showing the pyrene-based compounds having a benzothiazolium group of the present invention having the ability to visualize lysosomes in live cells. The LC₅₀ values for the pyrene-based compounds having a benzothiazolium group of the present invention were calculated to be in the 25-30 mM range by using CellTiter-Glow Luminescent® cell viability assay in COS-7. These results further confirmed that these probes are suitable for biological cell studies since the working concentration of the probes are far below their calculated LC₅₀ values. Bright fluorescent confocal microscopy images could be obtained when the concentration of the pyrene-based compounds having a benzothiazolium group of the present invention was as low as 100 nM.

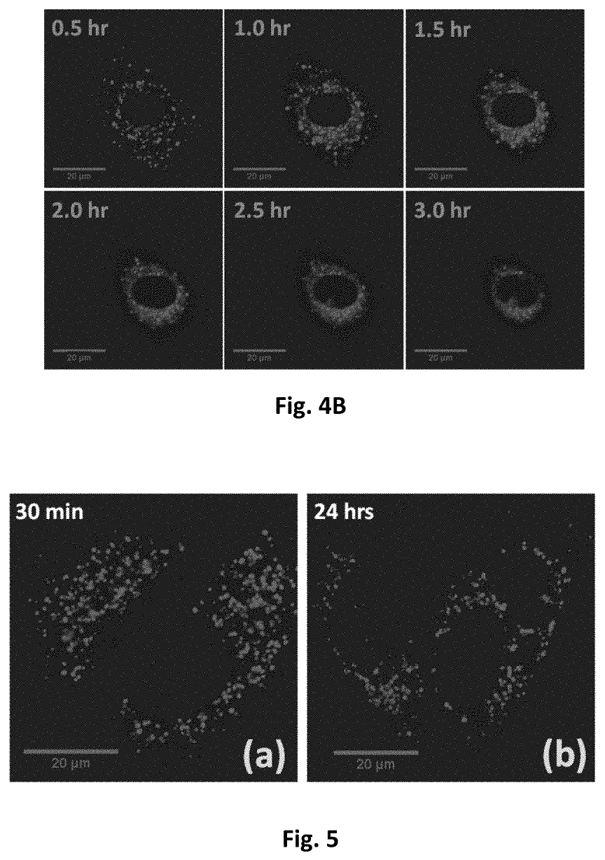

The lack of cellular pH perturbation, in addition to its low toxicity, raised the possibility of using the pyrene-based compounds having a benzothiazolium group of the present invention for long term lysosome imaging. Thus, A549 cells were treated with a pyrene-based compound having a benzothiazolium group of the present invention (500 nM) over a period of 6 hours, which gave stable fluorescent confocal microscopy images as shown in FIG. 4A. A549 cells were first incubated with a probe made from the pyrene-based compounds having a benzothiazolium group of the present invention (500 nM) for 30 minutes. Then, images were obtained every 30 minutes under consistent parameters of the microscope. The probe made from the pyrene-based compounds having a benzothiazolium group of the present invention was excited with a 561 nm laser. In sharp contrast, cells stained with commercial LysoTracker® Red DND-99 showed significant morphological changes and notable cell shrinkage after 1.5 hours as shown in FIG. 4B. The fluorescence signals of the pyrene-based compounds having a benzothiazolium group of the present invention and LysoTracker® Red was further analyzed by plotting their average fluorescence intensity vs. staining time and no apparent intensity changes were observed from the pyrene-based compound having a benzothiazolium group of the present invention in A549 cells over a period of 6 hours.

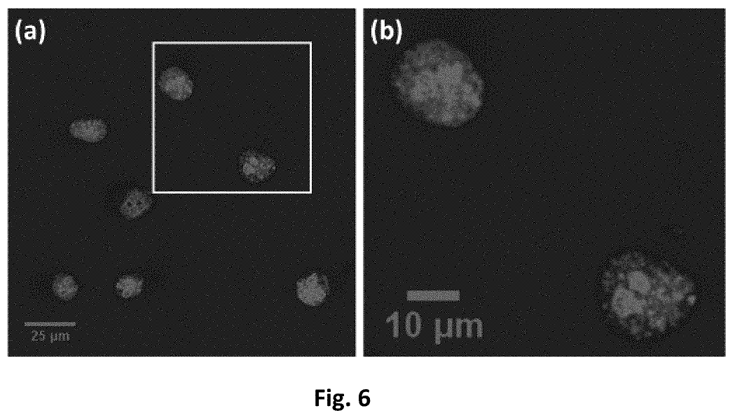

However, LysoTracker® exhibited a significant decrease in relative intensity with time, which could be attributed to the fluorescence quenching due to pH elevation within lysosomes (i.e. alkalinizing effect). In another experiment, COS-7 cells were incubated with a pyrene-based compound having a benzothiazolium group of the present invention for 30 minutes and 24 hours, and a clear fluorescence signal was still observable even after 24 hours in comparison with initial confocal microscopy images as shown in FIG. 5. The ability of the pyrene-based compounds having a benzothiazolium group of the present invention in giving stable fluorescence signals in intracellular environment opens the possibility for long term imaging, due to elimination of pH sensitivity.

Photostability was evaluated by studying samples of A549 cells that were stained with pyrene-based compounds having a benzothiazolium group of the present invention or LysoTracker® Red for 30 minutes separately. The resulting samples were continuously irradiated with a 561 nm laser. The fluorescence intensity, was plotted as a function of irradiation time. The pyrene-based compounds having a benzothiazolium group of the present invention exhibited excellent stability in comparison with the commercial LysoTracker® Red, without showing significant photobleaching during the experiment period. These result also suggest that the substituent on the benzothiazolium segment has a large impact on the photostability of the pyrene-based compounds having a benzothiazolium group of the present invention.

Since the structure of the pyrene-based compounds having a benzothiazolium group of the present invention do not include a basic amino group, the inventors questioned how the pyrene-based compounds having a benzothiazolium group of the present invention accumulate in cellular lysosomes. Since both pyrene and benzothiazolium segments are quite inert to protonation, the acidic environment in the organelle might not promote accumulation of the pyrene-based compounds having a benzothiazolium group of the present invention in the lysosome lumen. It is thought that the hydrophobic nature of the pyrene segment of the pyrene-based compounds having a benzothiazolium group of the present invention causes the pyrene-based compounds having a benzothiazolium group of the present invention to enter hydrophobic regions (i.e., lysosomal membrane or lysosomal proteins) rather than the aqueous lysosomal lumen. In order to evaluate the hypothesis, generated lipid vesicles were generated in vitro that mimic an artificial phospholipid bilayer structure similar to cellular membranes. In the control experiment, the pyrene-based compounds having a benzothiazolium group of the present invention did not show any appreciable fluorescent signal in aqueous HEPES buffer (pH=7.4).

Fluorescence of the pyrene-based compounds having a benzothiazolium group of the present invention in different pH aqueous solutions revealed that emission was only slightly affected (about ±10%) in a wide pH range. Since the fluorescence of the pyrene-based compounds having a benzothiazolium group of the present invention does not change significantly from a neutral pH to an acidic pH, it is believed that the dye is binding to a hydrophobic region of lysosomes. In another experiment, an acidic aqueous solution of (pH=4.5) a pyrene-based compound having a benzothiazolium group of the present invention was titrated with 10% bovine serum albumin (BSA). The addition of BSA led to a significant fluorescent enhancement of about 600%. It should be pointed out that BSA binding did not cause fluorescence enhancement in commercial LysoTracker® probes, since the PET effect from an amine group would be stronger in BSA than in an acidic aqueous solution. Therefore, interaction mechanism of the pyrene-based compounds having a benzothiazolium group of the present invention in cellular lysosomes should be significantly different from that of the acidotropic commercial LysoTracker® probes.

### Experiment 2

1 mmol of a 1-alkyl-4-methylpyridinium salt was added to 1.2 mmol pyrene-1-carboxaldehyde in 15 mL of ethanol. The solution was stirred at room temperature for 5 minutes. Then, 0.5 mL of pyridine was added so as to promote the reaction. This solution was heated to 65° C. and stirred for 48 hours. After completion of the reaction, the mixture was cooled down to room temperature and concentrated under vacuum. To the resulting orange-brown colored solid, 20 mL of ethyl acetate was added and the solution was left to settle for 30 minutes. Upon addition of ethyl acetate, an orange colored precipitate started to form at the bottom of the flask. Then, the brown colored solid was collected by vacuum filtration and washed with 10 mL of ethyl acetate three times. This experiment was done multiple times with various pyridinium salts and the final products were collected as a fine orange powder with about a 62% yield.

### Cell Culture, Transfection and Cell Treatments

For the cell culture, transfection and cell treatment aspects of this experiment, COS-7 cells were maintained in DMEM containing 1% FBS and 1% Penstrep at 37° C. in a 5% CO₂ humidified incubator. The cells were grown to 70 to 80% confluence before passing or transfection. Transfections were performed using Lipofectamine 2000 reagent according to the manufacturer's protocol. Stock solutions of the pyrene-based compounds having a pyridinium group of the present invention were made in DMSO. For live cell imaging, cells were treated with 500 nM EPP in PBS for 30 minutes at 37° C. following 14 hours post-transfection with pm Turquoise2-H2A. Final DMSO percentage was less than 0.25% (V/V). For fixed cell imaging, cells were first fixed by incubating in ice cold 100% methanol at −20° C. for 15 minutes. Following cell fixation, cells were then washed three times with PBS for 5 minutes and then were treated with 1 μM of a solution made from the pyrene-based compounds having a pyridinium group of the present invention for 1 hour at 37° C.

### Fluorescence Confocal Microscopy

For the fluorescence confocal microscopy aspect of the invention, COS-7 cells were imaged by using a Zeiss LSM 710 fluorescence confocal microscope with an oil 63×1.4 numerical aperture objective. For the nuclear co-localization experiments, pm Turguoise2-H2A transfected cells were excited with a 454 nm laser line and the emissions were collected from the 465 nm to 530 nm range. Solutions made from the pyrene-based compounds having a pyridinium group of the present invention were excited with a 488 nm laser line utilizing a one-photon absorption process and emissions were collected from the 560 nm to 700 nm range. Co-localization experiment results were further analyzed by using Zeiss LSM software. Values greater than 0.6 confirm the co-localization of the two fluorophores.

### Cell Viability Testing

For the cell viability testing aspect of the invention, COS-7 cells were seeded in Corning™ 96 clear well bottom, tissue culture opaque microplates. Approximately 1000 cells were plated in each well (cell density=1×10⁴ cells/ml). 24 hours after seeding, cells were treated with increasing concentrations of dyes made from solutions of the pyrene-based compounds having a pyridinium group of the present invention for 1 hours at 37° C. Cell toxicity assays were performed using CellTiter-Glo Luminescent® cell viability assay kits (Promega) according to the manufacturer's protocol. Luminescence readings were recorded using Spectramax® 5e microplate readers by using the default software installed. LCSO values for the dyes were calculated by using dose-response formulas in Origin8 software.

### Results

The pyrene-based compounds having a pyridinium group of the present invention exhibited absorption in dichloromethane at about 477 nm, which is notably shifted from pyrene-based compounds lacking a pyridinium group (about 350 nm). The fluorescence of the pyrene-based compounds having a pyridinium group of the present invention exhibited intense red emission peaks at about 610 nm (ϕ_{fl}≈0.41 in CH₂Cl₂). Therefore, probes made from the pyrene-based compounds having a pyridinium group of the present invention exhibit the desirable optical properties for biological cell studies, which include high fluorescence quantum yield, red emission with a large Stokes shift (Δλ≈133 nm), and an excitation maximum near 488 nm (one of the laser frequencies used in a confocal microscope). The emission intensity of the probes made from the pyrene-based compounds having a pyridinium group of the present invention was also stable and not affected in the pH range of 1-12.

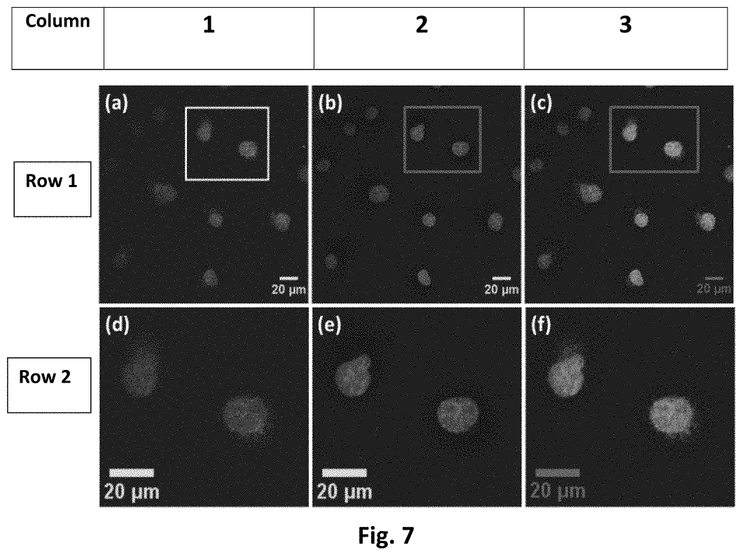

The attractive photophysical characteristics, red emission with a large Stokes shift and excitable at 488 nm laser frequency, indicates that dyes made from the pyrene-based compounds having a pyridinium group of the present invention might be useful for biological cell imaging. Thus COS-7 cells were stained with the pyrene-based compounds having a pyridinium group of the present invention and examined under a confocal microscope. When the stained cells were excited at 488 nm, the pyrene-based compounds having a pyridinium group of the present invention were found to accumulate on the cell nucleus, exhibiting remarkable selectivity to give bright red fluorescence as shown in FIG. 6.

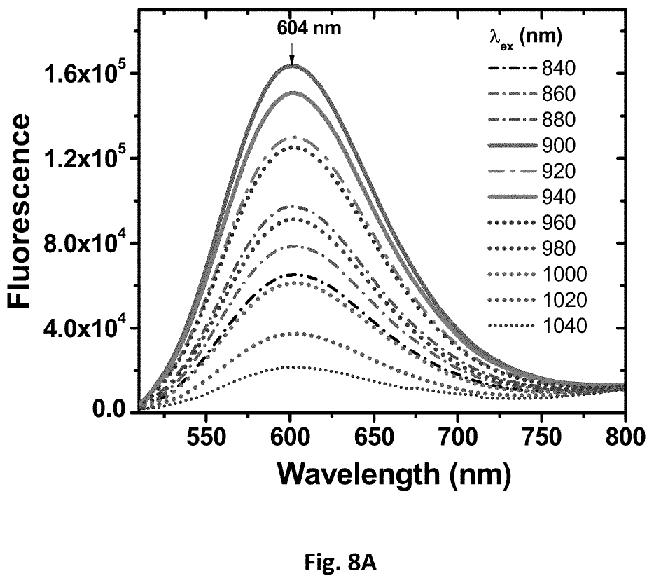

The ability of the pyrene-based compounds having a pyridinium group of the present invention for selective nucleus staining was further examined by using the pmTurquoise2-tagged H2A protein that is highly fluorescent and specific for nuclear localization. Co-localization studies were performed by transfecting COS-7 cells to express the pmTurquoise2-tagged H2A protein for nuclear localization, while the cells were co-stained with the pyrene-based compounds having a pyridinium group of the present invention as shown in FIG. 7. Column 1 shows fluorescent confocal microscopy images of COS-7 cells stained with commercial pmTurquoise-H2A for 14 hours; column 2 shows fluorescent confocal microscopy images of COS-7 cells stained with a probe made from pyrene-based compounds having a pyridinium group of the present invention; column 3 shows overlapped images of the images of column 1 with the images of column 2; and the images of row 2 show a digitally magnified view of the selected region in the corresponding image of row 1. The co-localization study manifested the nuclear selectivity of the pyrene-based compounds having a pyridinium group of the present invention. Although pyridinium-containing cyanine dyes often target mitochondria, probes made from the pyrene-based compounds having a pyridinium group of the present invention did not show any detectable mitochondrial localization, as repeated tests showed only the cell nucleus being targeted.

In addition, application of the pyrene-based compounds having a pyridinium group of the present invention does not require post-staining washing, as its fluorescence was turned-on upon entering the cell nucleus. This “wash-free” feature allows for convenient nucleus imaging immediately after the incubation (without the need for removing the excess dyes). After verifying the nucleus staining on live cells, probes made from the pyrene-based compounds having a pyridinium group of the present invention were further examined on fixed cells. COS-7 cells were first transfected to express the pmTurquoise-H2A tagged protein and then fixed with methanol. Fixed cells were incubated with probes made from the pyrene-based compounds having a pyridinium group of the present invention (1 mM) for one hour before imaging. Confocal microscope images showed satisfactory co-localization within the cell nucleus which further confirmed selective nuclear staining.

The pyrene segment of the pyrene-based compounds having a pyridinium group of the present invention is thought to play an important role in the interaction of probes made with the pyrene-based compounds having a pyridinium group of the present invention with DNA, as planar aromatic compounds are usually regarded as DNA intercalators. In order to shed some light on the interaction between probes made from the pyrene-based compounds having a pyridinium group of the present invention and DNA, fluorescence spectra were acquired when DNA was added into probes made from the pyrene-based compounds having a pyridinium group of the present invention in a PBS buffer solution (pH=7.4). Initial addition of DNA (<25 μg mL⁻¹) caused partial fluorescence quenching of the probes made from the pyrene-based compounds having a pyridinium group of the present invention in an aqueous solution, which was also observed for DRAQ5. Although the interaction between probes made from the pyrene-based compounds having a pyridinium group of the present invention and DNA could cause the accumulation of a dye on DNA, the observed partial fluorescence quenching during the initial DNA titration of probes made from the pyrene-based compounds having a pyridinium group of the present invention was not due to the simple change in local dye concentration, as a control experiment in aqueous solution showed that the fluorescence intensity of probes made from the pyrene-based compounds having a pyridinium group of the present invention constantly increases with the dye's concentration in the absence of DNA. Therefore, the initial fluorescence quenching was attributed to the interaction between probes made from the pyrene-based compounds having a pyridinium group of the present invention and DNA, possibly involving insertion of dye molecules into DNA groves. The requirement of using less DNA for the induction period suggests that probes made from the pyrene-based compounds having a pyridinium group of the present invention could interact with DNA more efficiently than DRAQ5.

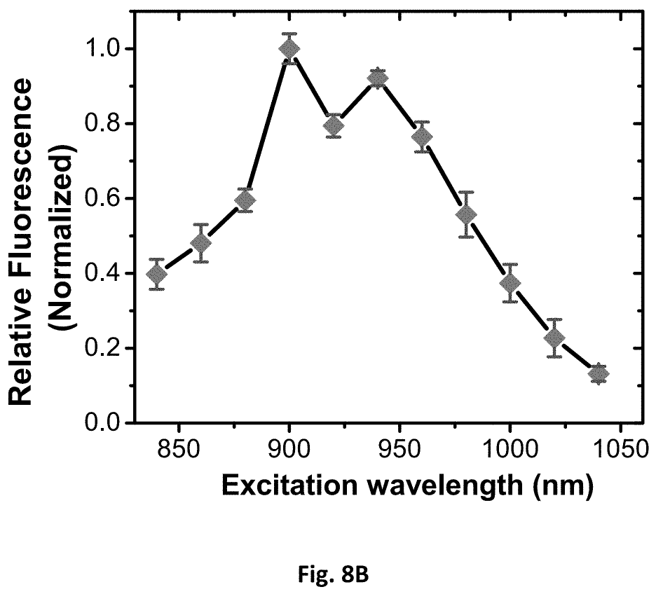

The pyrene-based compounds having a pyridinium group of the present invention can be excited via a two-photon absorption process. The pyrene-based compounds having a pyridinium group of the present invention exhibited one-photon absorption in dichloromethane at about 477 nm, and intense red emission peaks at about 610 nm. This raises the possibility of exciting the compound of the present invention via two-photon absorption at about 950 nm, which is near the desired NIR window (700-900 nm) for biological imaging applications. By exciting imaging reagents at the NIR window (with lower energy photons), two-photon fluorescence microscopy has a distinct advantage in minimizing possible photodamage and photobleaching of the tissues and organelles, and thus allowing the excitation photons to reach deep tissue. The pyrene-based compounds having a pyridinium group of the present invention exhibited emission in CH₂Cl₂ solution via two photon absorption at about 899 nm and 940 nm as shown with FIGS. 8A and 8B). Two-photon microscopy images of COS-7 cells stained with a probe made from pyrene-based compounds having a pyridinium group of the present invention, after 30 minutes incubation, exhibited the bright imaging for cellular nuclei upon excitation with 940 nm laser.

In light of the foregoing, it should be appreciated that the present invention significantly advances the art by providing a pyrene-based compound for imaging intracellular organelles that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A pyrene-based compound for imaging intracellular organelles, where the pyrene-based compound is defined by a formula selected from the group consisting of: 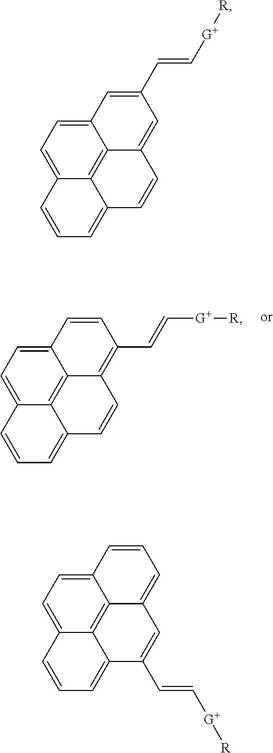
wherein G⁺ is selected from the group consisting of a pyridinium or a benzothiazolium group and wherein R is selected from the group consisting of a methyl group, an ethyl group, a propyl group or a benzyl group.

2. The pyrene-based compound of claim 1 wherein the pyridinium group is selected from the group consisting of 4-alkylpyridinium or 2-alkylpyridinium.

3. The pyrene-based compound of claim 2 wherein the pyrene-based compound is used to image a nucleus of a cell.

4. The pyrene-based compound of claim 1 wherein the benzothiazolium group is selected from the group consisting of 3-alkylbenzothiazolium, 3-alkylbenzooxazolium, 1-methyl-3alkylbenzoimidazolium, or 3,3-dimethyl-1-alkylindolium.

5. The pyrene-based compound of claim 4 wherein the pyrene-based compound is used to image the lysosomes of a cell.

6. The pyrene-based compound of claim 1 wherein the pyrene-based compound exhibits a bright red emission of between about 600 nm and about 650 nm when excited via a one-photon absorption process at about 475 nm.

7. The pyrene-based compound of claim 1 wherein the pyrene-based compound exhibits a bright red emission of between about 600 nm and about 650 nm when excited via a two-photon absorption process upon excitation by about a 950 nm laser.

8. The pyrene-based compound of claim 1 wherein the pyrene-based compound has a Stokes' shift of greater than about 130 nm.

9. The pyrene-based compound of claim 1 wherein the pyrene-based compound has a LC₅₀ of at least 8 μM.

10. A method of producing a pyrene-based compound, the method comprising the steps of:
a. adding a benzothiazolium salt or pyridinium salt to a pyrene-carbaldehyde to form a solution;
b. adding a reaction promotor to the solution; and
c. heating the solution so as to produce a pyrene-based compound defined by a formula selected from the group consisting of: 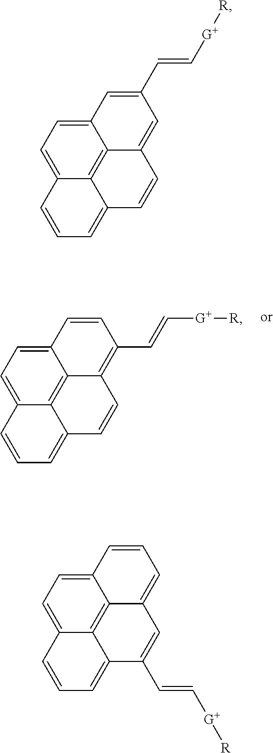
wherein G⁺ is selected from the group consisting of a pyridinium or a benzothiazolium group and wherein R is selected from the group consisting of a methyl group, an ethyl group, a propyl group or a benzyl group.

11. The method of claim 10 wherein a benzothiazolium salt is utilized and the benzothiazolium salt is selected from the group consisting of 3-alkyl-2-methylbenzothiazol-3-ium halides.

12. The method of claim 11 wherein the alkyl group is selected from the group consisting of methyl, ethyl, propyl, and benzyl; and wherein the halide group is selected from the group consisting of chloride, bromide, and iodide.

13. The method of claim 10 wherein a pyridinium salt is utilized and the pyridinium salt is selected from the group consisting of 1-alkyl-4-methylpyridinium halides.

14. The method of claim 13 wherein the alkyl group is selected from the group consisting of methyl, ethyl, propyl and benzyl; and wherein the halide group is selected from the group consisting of chloride, bromide, and iodide.

15. The method of claim 10 wherein the pyrene-carbaldehyde is selected from the group consisting of pyrene-1-carboxaldehyde, pyrene-2-carboxaldehyde, and pyrene-4-carboxaldehyde.

16. The method of claim 10 wherein the reaction promoter is selected from the group consisting of pyridine, piperidine, and triethylamine.
